(19) United States
(12) Patent Application Publication
(10) Pub. No.: US 2007/0032873 A1
(43) Pub. Date: Feb. 8, 2007

(54) Total artificial intervertebral disc

(75) Inventor: Madhavan Pisharodi, Brownsville, TX (US)

Correspondence Address: Mark R. Wisner;WISNER & ASSOCIATES, Suite 400, 1177 West Loop South, Houston, TX, 77027-9012, US

(73) Assignee: Perumala Corporation, Brownsville, TX (US)

(21) Appl. No.: 11/246,981

(22) Filed: Oct. 7, 2005

Related U.S. Application Data
(63) Continuation-in-part of application No. 11/195,880, filed on Aug. 2, 2005.

(51) Int. Cl. A61F 2/44

(52) U.S. Cl. 623/17.12; 623/17.13

(57) ABSTRACT

An artificial intervertebral disc that uses two orthogonally-oriented cushions, one with a greater height than width and the other with a greater width than height, comprised of a resilient material and affixed to each other in a manner to resist relative movement therebetween, to maintain the spacing between the vertebrae adjacent an intervertebral disc, to distribute and cushion against compression loads, and to mimic the normal kinematics of the intact, healthy intervertebral disc. One of the two cushions at least partially surrounds a frame that both provides resistance to compression and tension loads and translates the axis of rotation of the spinal column anteriorally and posteriorally as the patient bends and rotates.

### BACKGROUND OF THE INVENTION

This application in a continuation-in-part of co-pending application Ser. No. 11/195,880, filed Aug. 2, 2005, entitled ARTIFICIAL INTERVERTEBRAL DISC.

The present invention relates to an artificial disc that does not include a joint or sliding portions, but still maintains the flexibility of the spine, as well as the cushioning effect of the disc, after surgical replacement of a disc. In more detail, the present invention relates to an artificial disc for use in surgical replacement of an intervertebral disc that retains the properties of cushioning and resistance to flexure of the spine, as well as allowing the normal range of motions that characterizes the healthy intervertebral disc.

The injured, deformed, diseased, and/or degenerated human spine is a source of great pain in many patients, and there are many approaches to management, treatment, and/or prevention of that pain, including surgical intervention. One particularly vexing source of spinal pain and/or dysfunction is the damaged intervertebral disc. Healthy intervertebral discs are a necessity to pain-free, normal spinal function, yet disc function is all too frequently impaired by, for instance, disease or injury.

The anatomy of the intervertebral disc correlates with the biomechanical function of the disc. The three major components of the disc that are responsible for the function of the disc are the nucleus pulposus, annulus fibrosus, and cartilagenous endplate. The nucleus pulposus is the centrally located, gelatinous network of fibrous strands, surrounded by a mucoprotein gel, that prevents buckling of the annulus and maintains the height of the disc (and therefore, provides the cushioning effect and resistance to spinal flexure that are so important to spinal function) through osmotic pressure differentials. The water content of the disc changes in accordance with the load on the spine, water being driven out of the pulposus under heavy load. The annulus fibrosus encapsulates the disc, resisting both tension and compression loads and bearing axial loads. The vertebral endplates are cartilagenous in nature and “sandwich” the other components of the disc, distributing load over the entire disc and providing stability during normal spinal movements. The three elements work in cooperative fashion to facilitate disc function, and impairment of any of the elements compromises the functions of the other elements.

The two main surgical treatments of the intervertebral disc include total disc and nuclear replacement, but unfortunately, both treatments represent a number of compromises that simply do not provide normal disc function. The total artificial disc prosthesis is a total prosthetic replacement of the annulus fibrosus and nucleus pulposus with an endplate that interfaces with the patient's own vertebral endplates. Capturing and securing the total disc prosthesis to the host vertebral endplates can be a challenge because of the asymmetrical and cyclic loads placed upon the spine that can place excessive stresses on both the host bone and the interface between the prosthesis and the endplates, resulting in early loss of fixation. Many presently available total disc prostheses are designed to mimic the function of normal joints, but in that aspect, they are non-physiological in the sense that the normal spine does not have actual joints or sliding functions, but does have an inherent shock absorbing function. This lack of cushioning and shock absorbing function may be the contributing factor for the settling of the prosthesis into the vertebral body. For a summary of some of the disadvantages and limitations of known disc replacements, reference may be made to C. M. Bono and S. R. Garfin, History and Evolution of Disc Replacement, The Spine Journal, Vol. 4, pp. 145S-150S (2004) and E. G. Santos, et al., Disc Arthroplasty: Lessons Learned from Total Joint Arthroplasty, The Spine Journal, Vol. 4, pp. 182S-189S (2004).

Nuclear replacement is intended to replace a damaged nucleus pulposus with a device that is intended to restore disc height while maintaining the kinematics of the gel that comprises the healthy, intact nucleus pulposus. Although less invasive of the spine, implant extrusion and migration of the implant are all too frequent complications of nuclear replacement surgery. Some of the disadvantages and limitations of known devices for disc replacement are summarized in C. M. Bono and S. R. Garfin, History and Evolution of Disc Replacement, The Spine Journal, Vol. 4, pp. 145S-150S (2004) and in A. N. Sieber and J. P. Kostuik, Concepts in Nuclear Replacement, The Spine Journal, Vol. 4, pp. 322S-324S (2004).

It is, therefore, an object of the present invention to provide a total artificial intervertebral disc that is intended to overcome the disadvantages and limitations of these prior art devices comprising a frame, first and second cushions, one of which partially surrounds the frame, and means for resisting relative movement between the two cushions. The frame is provided with means for selectively engaging the vertebrae adjacent the intervertebral disc space when the artificial intervertebral disc is inserted into the space between two adjacent vertebrae, thereby resisting anterior-posterior movement of the artificial disc relative to the adjacent vertebrae.

Another object of the present invention is to provide a total artificial disc that maintains the normal range of motion of the spine and provides a cushioning function that approximates the normal function of the intervertebral disc under compression load.

Another object of the present invention is to provide a total artificial disc that is comprised of three main components that together function to provide the cushioning provided by cooperation of the three components of the normal intervertebral disc.

Another object of the present invention is to provide a total artificial disc in which the axis of rotation translates in the anterior-posterior direction in a manner that approximates normal disc function.

Another object of the present invention is to provide a total artificial disc that is adapted for use in adjacent segments of the spine.

Other objects, and the many advantages of the present invention, will be made clear to those skilled in the art in the following detailed description of several preferred embodiments of the present invention and the drawings appended hereto. Those skilled in the art will recognize, however, that the embodiments of the invention described herein are only examples provided for the purpose of describing the making and using of the present invention and that they are not the only embodiments of artificial discs that are constructed in accordance with the teachings of the present invention.

### SUMMARY OF THE INVENTION

The present invention addresses the above-described problem by providing an artificial intervertebral disc comprising an artificial intervertebral disc comprising a frame, a first resilient cushion having a greater height than width surrounding a portion of the frame, and a second resilient cushion having a greater width than height. Means is formed on the first cushion, the second cushion, or both the first and second cushions, for affixing the first and second cushions to each other and resisting relative movement therebetween with the width of the second cushion being oriented substantially orthogonally to the height of the first cushion.

In another aspect, the present invention provides a method of mimicking the function of the intervertebral disc of the intact spinal column after removal of some or all of the intervertebral disc from between the two adjacent vertebrae comprising the steps of inserting a first resilient cushion having a height greater than its width and a notch formed in one end thereof into the intervertebral disc space with the height of the first cushion oriented substantially parallel to the longitudinal axis of the spinal column, anchoring a frame surrounded at least in part by the first resilient cushion to one or both of the vertebrae adjacent the intervertebral disc space, inserting a second resilient cushion having a width greater than its height into the notch of the first resilient cushion with the width of the second cushion oriented orthogonally to the height of the first cushion, and resisting relative movement between the first and second cushions.

### BRIEF DESCRIPTION OF THE DRAWINGS

Referring now to the figures, FIG. 1 shows a perspective view of one embodiment of an artificial intervertebral disc constructed in accordance with the teachings of the present invention.

FIG. 2 is a perspective view of a frame that comprises one component of the artificial intervertebral disc of FIG. 1.

FIG. 3 is an exploded, perspective view of one of the two cushions and the fork comprising the artificial intervertebral disc of FIG. 1.

FIG. 4 is an exploded, perspective view of the second of the two cushions and the saddle comprising the artificial invertebral disc of FIG. 1.

FIG. 5 an exploded, perspective view of an alternative embodiment of the artificial intervertebral disc of the present invention.

### DETAILED DESCRIPTION OF THE PREFERRED EMBODIMENT(S)

In more detail, FIG. 1 shows a first embodiment of an artificial disc constructed in accordance with the teachings of the present invention at reference numeral 10. Artificial disc 10 is comprised of three main components, each described in more detail below, a frame 12, first cushion 14, and second cushion 16, each of first and second cushions 14 and 16 being provided with a cavity 18 formed therein. A fourth component is the screw 20 that extends through each of frame 12 and first and second cushions 14, 16. Although shown in the figures in a configuration that reflects the use of the artificial dic 10 for replacement of an intervertebral disc in the cervical regions of the spine, those skilled in the art will recognize from the following description that, with appropriate changes in size and configuration, the artificial disc of the present invention can also be utilized to advantage for total disc replacement in other portions of the spine.

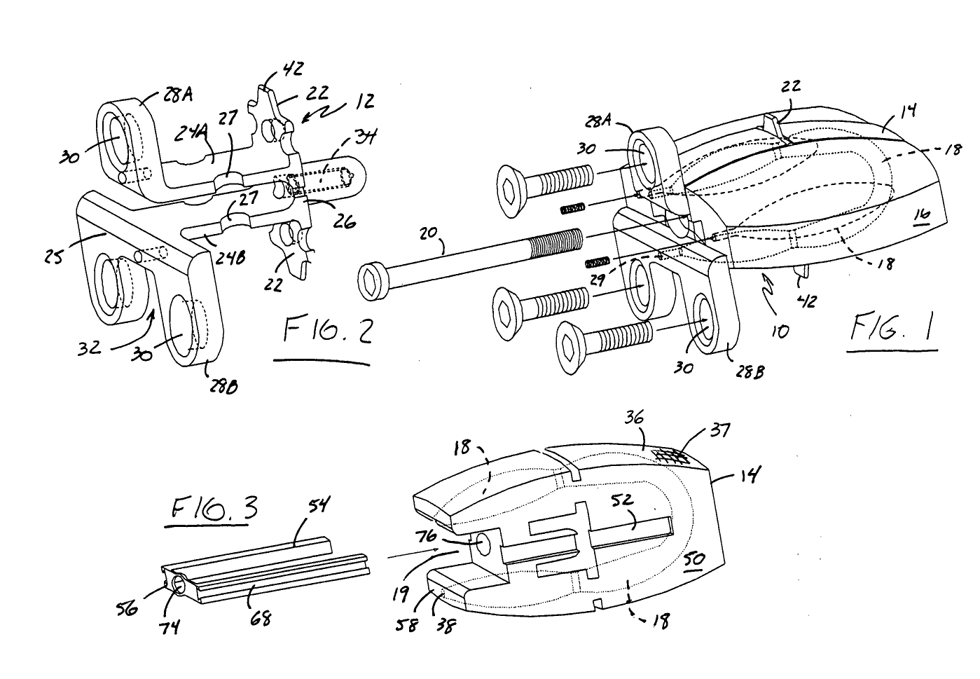

Frame 12 is better illustrated in FIG. 2, and by reference to that figure, it can be seen that frame 12 is comprised of two spaced apart arms 24 connected at one end by a bridge 26. One or both of the ends 25 of the arms 24 opposite bridge 26 are provided with ears 28 having one or more holes 30 formed therein for receiving one or more screws 31 (see FIG. 1) for securing frame 12 to the bodies of the vertebrae (not shown) adjacent the intervertebral disc space into which artificial disc 10 is inserted. In the preferred embodiment shown, both ends of arms 24 are provided with ears 28, the ear 28A on the end of one arm 24A being shaped in the form of a “U” and having two holes 30 formed therein, the portion of ear 28A between the holes 30 being cutout at 32 to form the arms of the “U”-shaped ear 28A, and the ear 28B on the end of the other arm 24B being shaped in the form of an inverted “U” and having a single hole 30 therein. This arrangement of “u” and inverted “U”-shaped ears 28A and 28B allows the use of the artificial disc 10 of the present invention in the intervertebral disc spaces of successive segments of the spinal column. When secured to the body of the adjacent vertebra, the inverted “U”-shaped ear 28B of one artificial disc extends into the cutout portion 32 of the “U”-shaped ear 28A secured to the body of that same vertebra. As best shown in FIG. 2, the bridge 26 of frame 12 is provided with a hole 34 for a purpose to be made clear below.

In the preferred embodiment, frame 12 is comprised of a material that tends to return to its original shape after the frame is subjected to either a compression or tension load. In other words, when the disc 10 is inserted into the intervertebral disc space, it is subjected to both compression and tension loads as the spine flexes and as the patient moves during his/her normal daily routine, and when subjected to compression and tension loads, the frame deforms. Under compression, the ends 25 of the arms 24 opposite bridge 26 tend to move closer to each other and when in tension, the ends 25 of the arms 24 opposite bridge 26 tend to move further apart; in other words, the arms 24 of frame 12 deviate from their original spaced apart position (in the preferred embodiment shown, the two arms are substantially parallel, but those skilled in the art who have the benefit of this disclosure will recognize that the invention is not limited to a frame having parallel arms) when under compression or tension force. When the respective compression or tension force is relieved, the frame 12 tends to return to its original shape, i.e., the ends 25 of arms 24 opposite bridge 26 return to their original spaced relationship, and the arms assume their original, spaced apart relationship. When subjected to loads in this manner, frame 12 acts as both a “backbone” and as a spring to help both bear compression loads and relieve tension loads in a manner that mimics normal disc function. Note also that, when the artificial disc 10 of the present invention is inserted into the intervertebral disc space, the bridge 26 of frame 12 is positioned posteriorally relative to the ends of arms 24 opposite bridge 26. The spring function of frame 12 is advantageous because, as the patient bends forward, the ends of arms 24 opposite bridge 26 are subjected to compression loads, and the further the patient bends, the more the material comprising frame 12 tends to resist the compression load, providing the spring function discussed above. Further, biomechanical studies of normal, healthy spines have shown that the axis of rotation (the weight-bearing center of the intervertebral disc) translates anteriorally and posteriorally as the spine flexes, and the variable resistance provided by this configuration and placement of frame 12 in the intervertebral disc space helps provide this normal front-to-back shift in the axis of rotation, so that the total artificial disc of the present invention replicates that shifting in the axis of rotation. Materials that are characterized by this spring-like function when formed into the frame 12 include, but are not limited to stainless steel, titanium and titanium alloys, cobalt-chrome (Co—Cr) alloys, cobalt-chromium-molybdenum (Co—Cr—Mo), and medical grade (inert) polymeric plastics such as polyethylene, all as known in the art. The cushions 14, 16 and the cavities in the cushions 14, 16 filled with hydrogel improve this quality in the total artificial disc 10.

As noted above, the cushion 14 is molded over frame 12 (best shown in FIG. 2), and from that description of the structural relationship of frame 12 and cushion 14, it can be surmised that cushion 12 is preferably molded from a resilient, polymeric material. Although not limited to these materials, in the preferred embodiment, cushion 14 is molded from a biocompatible, viscoelastic polymer such as silicone, a urethane such as a polycarbonate urethane, or a polyurethane. As best shown in FIG. 3, the first cushion 14 is molded with a profile that approximates the shape of the normal intervertebral disc space with a height H greater than the width W; it can be see that the top and bottom surfaces 36 of cushion 14 are arched so that the height of cushion 14 is greater in the center than at its ends. This shape of cushion 14 is referred to as being biconvex, e.g., both the top and bottom surfaces 36 of cushion 14 are convex in the anterior-posterior direction.

In the preferred embodiment shown, the top and bottom surfaces 36 of cushion 14 are convex in the anterior-posterior and side-to-side directions and are provided with a textured or grooved surface (shown schematically at reference numeral 37) to facilitate the ingrowth of bone onto the surfaces 36. In a particularly preferred embodiment, the surfaces 36 of cushion 14 are covered with a porous or roughened titanium coating and perhaps even a layer of calcium phosphate for this purpose; other suitable coatings/surfaces are known in the art and include titanium wire mesh, plasma-sprayed titanium, porous cobalt-chromium and bioactive materials such as hydroxyapatite and the aforementioned calcium phosphate. This component of the artificial disc 10 of the present invention functions in a manner similar to the function of the cartilage of the normal, healthy artificial disc.

As noted above, the central portion of cushion 14 is provided with a cavity 18 having a sac 16 contained therein. Although the cavity 18 shown in the figures is kidney-shaped so as to approximate the shape of the nucleus pulposus of a normal intervertebral disc, those skilled in the art who have the benefit of this disclosure will recognize that the cavity need not be shaped in this shape and that, depending upon the particular pathology that causes the disc replacement, it may even be advantageous to shape the cavity 18 differently in contemplation of varying kinematic characteristics. The sac 16 is at least partially filled with a hydrogel such as a polyvinyl alcohol (PVA), synthetic silk-elastin copolymers, polymethyl- or polyethylmethacrylate, polyethylene or polyacrylonitrile that absorbs water and increases in volume upon absorption of water, thereby functioning to maintain disc height in a manner similar to the manner in which the healthy disc maintains proper spacing between adjacent vertebrae. To facilitate the absorption of water, the sac 16 is comprised of a material that is permeable to water and the cushion 14 of artificial disc 10 may be provided with a plurality of holes or channels (not shown) for allowing water to pass through the material comprising cushion 14 and access the permeable sac 16 containing the hydrogel. Materials that may be used to advantage as the sac 16 include woven polyethylene, woven and non-woven biocompatible synthetic fibers and other materials as known in the art. Because the sac 16 is contained within cavity 18, the strength of the material comprising sac 16 is not as important as the ability of that material to contain the hydrogel and pass water into and out of the hydrogel in a manner that mimics the absorption of water by the healthy nucleus pulposus.

As best shown in FIG. 2, the disc 10 of the present invention is provided with ports 38 through which hydrogel can be added or removed from the sac 16 in the cavity 18 of cushion 14. These ports 38 are comprised of channels that extend from the sac 16 to the periphery of cushion 14, and are preferably located on the periphery of cushion 14 adjacent the ears 28 of frame 12 since the disc 10 is implanted ventrally and the ears 28 therefore face the surgeon when the disc 10 is implanted in the intervertebral disc space, allowing access to ports 38 so that the surgeon can inject the hydrogel (or use a syringe to remove hydrogel) as needed to confer the desired amount of initial disc height to the implanted artificial disc. Once the desired disc height is obtained, the ports 38 are capped or plugged to prevent extrusion of the hydrogel contained within sac 16 or cavity 18. In an alternative embodiment, a one-way valve of a type known in the art may be utilized for this purpose.

As best shown in FIG. 1, first cushion 14 surrounds a portion of the frame 12, with the ends 25 of arms 24 and the ears 28 extending out of one end of cushion 14 and the bridge 26 of frame 12 being located within a notch 19 in cushion 14 that extends in a direction substantially parallel to the longitudinal axis of cushion 14. Frame 12 is provided with vertically-extending prongs 22 with points 42 that extend through the material comprising cushion 14 to engage and help the prongs 22 dig into the bone of the adjacent vertebrae (not shown), helping anchor disc 10 in the intervertebral disc space and resisting extrusion or shifting of the disc 10 relative to the adjacent vertebrae. To provide this arrangement in which the cushion 14 surrounds a portion of frame 12 with the ears 28 and the points 42 of prongs 22 extending beyond the end margin and/or periphery of the material comprising cushion 14, first cushion 14 is preferably molded over frame 12 or cast in place over frame 12. Contemplating the molding of cushion 14 over frame 12, by comparison of FIGS. 2 and 5, it can be seen that the arms 24A, 24B of frame 12 are provided with cutouts 27 that form ports that allow the material comprising cushion 14 to flow past the edges of arms 24A, 24B when in the mold so that a portion of the material comprising cushion 14 fills the notch 19 in cushion 14. In this manner, a portion of the material comprising cushion 14 is positioned between the arms 24 and proximate the bridge 26 of frame 12 to insure that frame 12 is retained in engagement with cushion 14.

The positioning of a portion of the material comprising cushion 14 between the arms 24 and proximate the bridge 26 of frame 12 serves additional purposes. As described above, the spring function of frame 12 provides not only resistance to compression and tension loads, but also the anterior-posterior translation of the axis of rotation as the spine flexes so as to mimic the kinematics of the healthy disc. First, because of the resilient nature of the material comprising cushion 14, the portion of the material comprising cushion 14 that is positioned between the arms 14 of frame 12 provides additional cushioning and resistance to the deformation of the frame 12 under extraordinary compression load. Second, because it is positioned proximate the bridge 26 of frame 12 and between the arms 24, the volume of the material comprising cushion 14 acts to regulate the amount of resistance to compression load as the patient bends. By positioning more of that material between the arms 24, the resistance to compression provided by frame 12 is increased, both because of the effective shortening of the length of the arms 24 and by the resistance to compression provided by that material itself. In the case of the molding of the cushion 14 over frame 12, the amount of material positioned between the arms 24 and proximate the bridge 26 of frame 12 is increased or decreased according to the size of the cutouts 27, which allow more or less of the material to flow into the notch 19. Of course the surgeon has the final discretion in fine-tuning the amount of resistance to the bending of arms 24 in the sense that some or all of the material positioned between the arms 24 can be trimmed from between the arms before the disc 10 is inserted into the intervertebral disc space.

As noted above, the central portion of cushion 14 is provided with a cavity 18 (FIGS. 1 and 3) in the material comprising the cushion that forms a horseshoe shape that extends from a first access port 38A to a second access port 38B. Those skilled in the art who have the benefit of this disclosure will recognize, however, that the cavity need not be shaped in this shape and that, depending upon such factors as the anatomy of the patient and the particular pathology that causes the disc replacement, it may even be advantageous to shape the cavity 18 differently in contemplation of varying kinematic characteristics. Although not separately numbered in the drawings since it is closely applied to the interior wall defining cavity 18 by injection of hydrogel (and therefore not distinguishable from the interior wall), the cavity 18 may optionally contain a sac for containing a hydrogel such as polyvinyl alcohol (PVA), synthetic silk-elastin copolymers, polymethyl- and/or polyethylmethacrylate, polyethylene or polyacrylonitrile that absorbs water and increases its volume upon absorption of water, thereby functioning to help maintain the disc height in a manner similar to the manner in which the healthy disc maintains proper spacing between adjacent vertebrae. To facilitate absorption of water, the sac is comprised of a material that is permeable to water and the cushion 14 of artificial disc 10 may be provided with a plurality of holes or channels (not shown) for allowing water to pass through the material comprising cushion 14 and access the permeable sac containing the hydrogel. Materials that may be used to advantage as the sac include woven polyethylene, woven and non-woven biocompatible synthetic fibers and other materials as known in the art. Because the sac is contained within cavity 18, the strength of the material comprising the sac is not as important as the ability of that material to contain the hydrogel and pass water into and out of the hydrogel in a manner that mimics the absorption of water by the healthy nucleus pulposus.

As best shown in FIG. 1, the cushion 14 is provided with ports 38 through which hydrogel can be added or removed from cavity 18. These ports 38 are comprised of channels that extend from the interior of cavity 18, or the interior of the sac contained therein (if the cavity 18 includes a sac) to the periphery of first cushion 14, and are preferably located on the periphery of cushion 14 adjacent the ears 28 of frame 12 since the disc is implanted ventrally and the ears 28 therefore face the surgeon when the disc 10 is implanted in the intervertebral disc space, allowing access to ports 38 so that the surgeon can inject the hydrogel (or use a syringe to remove hydrogel) as needed to confer the desired amount of disc height to the implanted artificial disc. Access ports 38 are coincident with the threaded bores 39 through the ears 28A, 28B of frame 12, and once the desired disc height is attained, the ports 38 are closed by inserting and tightening the threaded screws 41 in bores 39 to resist extrusion of the hydrogel contained within cavity 18 (or contained within sac 16 in cavity 18).

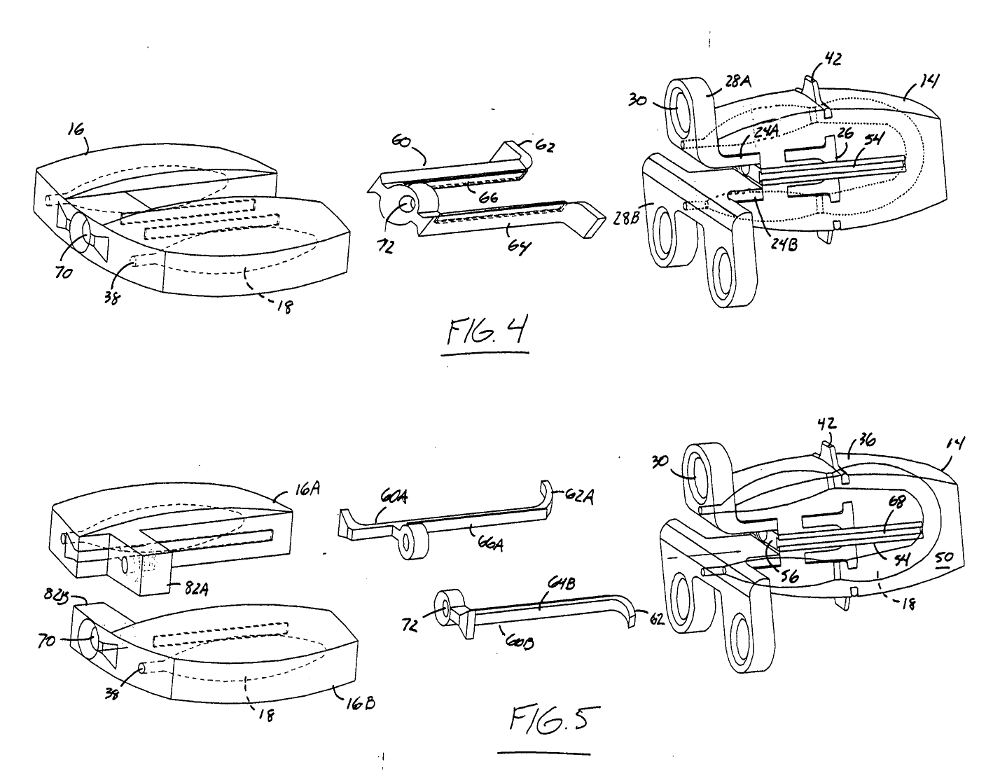

Referring now to FIG. 4, the second cushion 16 comprising the artificial intervertebral disc 10 is shown in more detail. Second cushion 16 is shaped similarly to first cushion 14, but because it is affixed to the first cushion 14 and frame at substantially a right angle to the height dimension of first cushion (which as noted above, is greater than the width), second cushion 16 is described as having a width that is greater than its height. Like first cushion 14, second cushion 16 is provided with an internal cavity 18, or more accurately, two internal cavities, for injecting hydrogel in the same manner and for the same purpose as described above in connection with first cushion 14. Access ports 38 allow the surgeon to inject (or remove) hydrogel as needed to confer the desired amount of disc height and resistance to compressive loads to the implanted artificial disc 10, it being recognized by those skilled in the art that the shape of the intervertebral disc space is such that second cushion 16, being dimensioned with a width greater than its height and being affixed to the frame 12 and first cushion 14 so that the width of second cushion 16 is oriented at approximately a right angle to the height of first cushion, so that the artificial disc 10 approximates the shape of the healthy intervertebral disc when implanted in the intervertebral disc space. More particularly, because the height of first cushion 14 is greater than its width and the height dimension is oriented in a direction substantially parallel to the axis of the spinal column when implanted in the disc space, first cushion 14 is shaped so as to contact the cortical bone of both the adjacent vertebrae even though the adjacent vertebrae are saucer-shaped so that the disc space is taller, or thicker at its center than at its periphery, and because the width of second cushion 16 is greater than its height and the width dimension is oriented in a direction substantially perpendicular to the axis of the spinal column when implanted in the disc space (and at approximately a right angle to the height dimension of first cushion 14), second cushion 16 is shaped so as to contact the adjacent vertebrae at the periphery of the disc space where the vertical dimension is minimal because of the saucer-shaped bearing surfaces of the adjacent vertebrae. Because of the shape of the artificial disc of the present invention, disc 10 provides a wide bearing surface over which compressive loads are distributed and stability, while still maintaining the necessary spacing between the adjacent vertebrae, when implanted in the disc space.

To affix the second cushion 16 to first cushion 14 in a manner that resists relative rotation therebetween, means is provided in the form of grooves 52 in the sides 50 of first cushion 14 for receiving the tines 54 of a fork 56 (FIG. 3) that is assembled to the end 58 of first cushion 14. As best shown in FIG. 5, the grooves 52 on the sides 50 of first cushion 14 are arranged so that when fork 56 is assembled to first cushion 14 (by sliding the tines 54 into the grooves 52), the fork 56 is positioned in the notch 19 of first cushion 14 between the two arms 24 of frame 12. In this manner, the fork 56 “captures” that portion of the material comprising first cushion 14 that is positioned between the arms 24 of frame 12 proximate bridge 26, thereby serving both to reinforce the material comprising cushion 14 from gross distortion due to compression loads and cooperating with the prongs 22 of frame 12 to resist movement of cushion 14 relative to frame 12. In much the same way that the fork 56 provides structural rigidity, resists deformation of first cushion 14, and restrains movement of first cushion 14 relative to frame 12, a saddle 60 (FIG. 4) is provided to function in a similar manner for second cushion 16. Second cushion 16 is preferably molded or cast over saddle 60, the stirrups 62 on the ends of the straps 64 of saddle 60 extend outwardly and are buried into the material comprising second cushion 16 so as to resist relative movement between the saddle 60 and second cushion 16. The inside surfaces of straps 64 are provided with raised keys 66 that engage complimentary-shaped grooves 68 formed in the outside surfaces of the tines 54 of fork 56 so that movement of the second cushion 16 relative to first cushion 14 is restrained by virtue of the interlock provided between the keys 66 on the inside surfaces of the straps 64 of saddle 60 and the grooves 68 on the outside surfaces of the tines 54 of fork 56. Of course those skilled in the art will recognize that the outside surfaces of the tines 54 of fork 56 could be provided with keys and the inside surfaces of the straps 64 of saddle 60.

It will also be apparent to those skilled in the art that the means formed on first cushion 14, second cushion 16, or both the first and second cushions 14, 16, for affixing the cushions 14, 16 to each other and resisting relative movement therebetween with the width of second cushion 16 being oriented substantially orthogonally to the height of first cushion 14 may also take the form of threaded bores and screws passing through the cushions 14, 16, sculpted or formed male projections and female receptacles on the cushions 14, 16 that lock the two cushions 14, 16 in place relative to each other,

As best shown in FIG. 1, a screw 20 extends from the periphery of cushion 16 through cushion 14 in an anterior-posterior direction along the longitudinal axis of the artificial disc 10. Screw 20 is inserted through the hole 70 in second cushion 16 and passes through the hole 72 formed in saddle 60, a similar hole 74 formed in fork 56, another hole 76 in the portion of the material comprising first cushion 14 that is positioned between the arms 24 of frame 12, and tightened onto the threads (not shown) in the blind bore 78 behind the bridge 26 of frame 12. When tightened onto the threads in bore 78, screw 20 places all of the component parts of the artificial disc of the present invention in a state of compression with the resilient material comprising each of first and second cushions 14, 16 “trapped” between the saddle 60, fork 56, and frame 12 to provide resistance to relative movement between the component parts, add structural rigidity, and because the resilient material comprising first and second cushions 14, 16 is under compression load, providing the desirable cushioning function that mimics the healthy intervertebral disc.

Referring now to FIG. 6, an alternative embodiment of the artificial intervertebral disc of the present invention is indicated generally at reference numeral 80. The frame and first cushion of the artificial disc 80 shown in FIG. 6 are the same as the frame 12 and first cushion 14 shown in FIGS. 1-5 and are therefore numbered with the same reference numerals. Second cushion 16, however, is partitioned into portions 16A and 16B along the longitudinal axis of second cushion 16 with overlapping tabs 82A and 82B for assembling the two portions 16A and 16B to each other around the two halves 60A and 60B comprising the saddle 60 of artificial disc 80. Construction of the second cushion 16 and saddle 60 in longitudinally divided portions facilitates the assembly of the artificial disc 80 at the time of surgery, even allowing the disc to be assembled in the intervertebral disc space, by sliding each of the two portions of second cushion 16 into place in the grooves 68 formed on the outside surfaces of the tines 54 of fork 56. To retain the two portions of second cushion 16 to the sides 50 of first cushion 14 during in situ assembly, the grooves 68 in the outside surfaces of the tines 54 of fork 56 are configured as a be recognized by those skilled in the art that to obtain desirable load resistance properties, it may be advantageous to make either or both of the cushions 14, 16 of a combination of materials, with an embedded layer of material having a second set of resilience and/or load-bearing characteristics, or as a laminated “sandwich” of polyurethane and other material(s), each material adding a unique component to the load bearing characteristics of the cushions 14, 16. All such changes, and others that will be clear to those skilled in the art from this description of the preferred embodiments of the invention, are intended to fall within the scope of the following, non-limiting claims.

What is claimed is:

1. An artificial intervertebral disc comprising:
a frame;
a first resilient cushion having a greater height than width surrounding a portion of said frame;
a second resilient cushion having a greater width than height; and
means formed on said first cushion, said second cushion, or both said first and second cushions, for affixing said first and second cushions to each other and resisting relative movement therebetween with the width of said second cushion being oriented substantially orthogonally to the height of said first cushion.

2. The artificial intervertebral disc of claim 1 additionally comprising a screw for holding said second cushion, said first cushion, and said frame to each other.

3. The artificial intervertebral disc of claim 1 additionally comprising a fork having tines extending along the sides of said first cushion between said first and second cushions.

4. The artificial intervertebral disc of claim 3 wherein said second cushion is provided with one or more grooves for interacting with one or both of the tines of said fork.

5. The artificial intervertebral disc of claim 4 additionally comprising a saddle interposed between said first and said second cushions.

6. The artificial intervertebral disc of claim 5 wherein the straps of said saddle interact with the tines of said fork.

7. The artificial intervertebral disc of claim 1 wherein said first cushion is provided with a cavity for receiving a hydrogel therein.

8. The artificial intervertebral disc of claim 1 wherein said second cushion is provided with a cavity for receiving a hydrogel therein.

9. The artificial intervertebral disc of claim 8 wherein said first cushion is also provided with a cavity for receiving a hydrogel therein.

10. The artificial intervertebral disc of claim 1 wherein said frame is comprised of spaced apart arms and a bridge connection said arms at one end thereof.

11. The artificial intervertebral disc of claim 10 wherein at least a portion of the material comprising said first cushion is positioned between the arms of said frame and proximate the bridge thereof.

12. The artificial intervertebral disc of claim 1 wherein one or both of said first and said second cushions is provided with central cavity having a sac therein, said sac containing a hydrogel.

13. The artificial intervertebral disc of claim 12 wherein said sac is comprised of a material that is permeable to water.

14. The artificial intervertebral disc of claim 12 additionally comprising an opening through said cushion for changing the amount of hydrogel within said sac.

15. The artificial intervertebral disc of claim 1 wherein said frame is comprised of a resilient material so that the ends of the arms opposite the bridge move toward and apart from each other in response to changes in load.

16. The artificial intervertebral disc of claim 1 wherein said first and said second cushions are comprised of polyurethane.

17. The artificial intervertebral disc of claim 1 wherein at least a portion of the surface of said first, said second, or both said first and said second cushions is provided with porous or roughened titanium, calcium phosphate, titanium wire mesh, plasma-sprayed titanium, porous cobalt-chromium, or hydroxyapatite.

18. A method of mimicking the function of the intervertebral disc of the intact spinal column after removal of some or all of the intervertebral disc from between the two adjacent vertebrae comprising the steps of:
inserting a first resilient cushion having a height greater than its width and a notch formed in one end thereof into the intervertebral disc space with the height of the first cushion oriented substantially parallel to the longitudinal axis of the spinal column;
anchoring a frame surrounded at least in part by the first resilient cushion to one or both of the vertebrae adjacent the intervertebral disc space;
inserting a second resilient cushion having a width greater than its height into the notch of the first resilient cushion with the width of the second cushion oriented orthogonally to the height of the first cushion; and
resisting relative movement between the first and second cushions.

19. The method of claim 18 additionally comprising compressing the first and second cushions.

20. The method of claim 18 wherein the frame comprises two arms connected at one end by a bridge and the method additionally comprises resisting the bending of the arms of the frame.
